(19) United States
(12) Patent Application Publication
(10) Pub. No.: US 2022/0002345 A1
(43) Pub. Date: Jan. 6, 2022

(54) Bone-Targeting Therapeutic Compositions and Methods of Using Them

(71) Applicant: Taithera Inc., New York, NY (US)

(72) Inventor: Mo CHEN, Forest Hills, NY (US)

(21) Appl. No.: 17/283,312

(22) PCT Filed: Oct. 8, 2019
(86) PCT No.: PCT/US2019/055102

§ 371 Date: Apr. 7, 2021

Related U.S. Application Data
(60) Provisional application No. 62/766,226, filed on Oct. 9, 2018.
(60) Provisional application No. 62/898,632, filed on Sep. 11, 2019.

(51) Int. Cl. C07K 7/08; A61K 31/704; A61P 35/00

(52) U.S. Cl. CPC C07K 7/08; A61K 38/00; A61P 35/00; A61K 31/704

(57) ABSTRACT

This invention provides a composition comprising (i) a peptide having an amino acid sequence that comprises the amino acid sequence TPLSYLKGLVTV and (ii) a therapeutic agent operably affixed thereto. This invention also provides related pharmaceutical compositions, kits, and methods for treating a subject afflicted with bone cancer.

This application claims the benefit of U.S. Provisional Applications No. 62/898,632, filed Sep. 11, 2019 and 62/766,226, filed Oct. 9, 2018, the contents of both of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to conjugates of therapeutic agents and bone-targeting peptides, and methods of using them to treat bone cancer.

### BACKGROUND OF THE INVENTION

Doxorubicin

Doxorubicin, sold under the trade names adriamycin, among others, is a chemotherapy medication used to treat various cancers. These cancers include breast cancer, bladder cancer, Kaposi's sarcoma, lymphoma, acute lymphocytic leukemia, Hodgkin's lymphoma, stomach cancer, lung cancer, ovarian cancer, thyroid cancer, soft tissue sarcoma, multiple myeloma, and others. Doxorubicin is given by injection into a vein. It is often used together with other chemotherapy agents. Commonly used doxorubicin-containing regimens are AC (adriamycin and cyclophosphamide), TAC (taxotere and AC), ABVD (adriamycin, bleomycin, vinblastine, and dacarbazine), BEACOPP, CHOP (cyclophosphamide, hydroxydaunorubicin, vincristine, and prednisone) and FAC (5-fluorouracil, adriamycin, and cyclophosphamide).

Common side effects include hair loss, bone marrow suppression, vomiting, rash, and inflammation of the mouth. Other serious side effects may include allergic reactions (such as anaphylaxis), heart damage, tissue damage at the site of injection, radiation recall, and treatment-related leukemia. People often experience red discoloration of the urine for a few days. Doxorubicin is in the anthracycline and antitumor antibiotic family of medications. It works in part by interfering with the function of a cancer cell's DNA.

Bone Metastases: Symptoms and Diagnoses

Each year, about 100,000 Americans with cancer find out that the cancer has spread to their bones. The bones are the most common place where metastatic breast cancer cells tend to go. For more than half of women who develop stage IV breast cancer, the bones are the first sites of metastasis. Although breast cancer can spread to any bone, the most common sites are the ribs, spine, pelvis, and long bones in the arms and legs.

Bone metastases are a major clinical concern that can cause severe pain, bone fractures, spinal cord compression, hypercalcemia, anemia, spinal instability, decreased mobility, and rapid degradation in the quality of life for patients. The most common bone metastases, or “bone mets”, are breast cancer, prostate cancer and ovarian cancer. Recently, the number of breast and colorectal cancer survivors has globally increased, and the 5-year survival rates are now ≤60% and ≥85%, respectively. In many countries, the 5-year survival rate for prostate cancer is ≥95%. An increase in the survival time increases the incidence of bone metastasis. Bone metastases caused by tumor cells leads to either osteoblastic or osteolytic phenotypes. Typically, osteolytic metastases are more aggressive than osteoblastic metastases. Regardless of the phenotype, though, bone metastases show osteoclast proliferation and hypertrophy. Recently, cancer chemotherapy has also made considerable progress in increasing survival rates of patients with far-advanced cancer.

The most common treatments for metastatic breast cancer in any location (bone, brain, lung, or liver) are systemic medications that treat cancer throughout the entire body. Systemic medications include chemotherapy, hormonal therapy, targeted therapies, and bone-strengthening medications. Often, bone metastases can be stabilized and managed for long periods of time.

Agents that target bone metastatic lesions, such as bisphosphonates (BPs) and denosumab (D-mab), have provided clinical benefits by preventing skeletal-related events (SER). However, an effective chemotherapy product for treating bone metastases is still not available.

An Unmet Need

The current approaches for treating bone metastases mainly include surgical resection, radiotherapy, and chemotherapy. Yet, their efficacies are greatly limited due to the low permeability of bone tissue and poor selectivity to the multiple bone metastatic nodules. There is thus an unmet need for a superior method of selectively delivering a therapeutic agent, particularly a chemotherapeutic, to bone tissue.

### SUMMARY OF THE INVENTION

This invention provides a composition comprising (i) a peptide having an amino acid sequence that comprises the amino acid sequence TPLSYLKGLVTV and (ii) a therapeutic agent operably affixed thereto.

This invention also provides a pharmaceutical composition comprising (i) the subject composition and (ii) a pharmaceutically acceptable carrier.

This invention further provides a method for treating a subject afflicted with bone cancer comprising administering to the subject a therapeutically effective amount of the subject composition.

Finally, this invention provides an article of manufacture (kit) comprising, in separate compartments, (a) one of, and ideally both of, (i) a diluent and (ii) a label instructing the user to administer a composition to a subject afflicted with bone cancer, and (b) the composition, wherein the composition comprises (i) a peptide having an amino acid sequence that comprises the amino acid sequence TPLSYLKGLVTV and (ii) a therapeutic agent operably affixed thereto.

### BRIEF DESCRIPTION OF THE FIGURES

FIG. 1

This figure shows the structure of NH₂—C(DOX-maleimidyl)-TPLSYLKGLVTVG-COOH.

FIG. 2

This figure shows the structure of NH₂-TPLSYLKGLVTVG-C(DOX-maleimidyl)-COOH.

FIG. 3

This figure shows that TRAP-Binding Peptide (TBP) directs a conjugated moiety to a tumor lesion site. NSG mice were inoculated with luciferase-labeled MDA-MB-231 breast cancer cell line (1 million cells) to the tibia. Three weeks after, 1 mg TBP-Texas red was injected intraperitoneally. 24 hours later, the animal was injected with luciferin, and the bioluminescence and fluorescence were monitored using an IVIS imaging stem. The vital organs were further dissected and imaged. Panels A and A′=Bioluminescence (breast cancer); and panels B and B′=TBP-Texas red. For panel A′, 1=leg with cancer cell inoculation, 2=collateral leg, 3=spleen, 4=lung, 5=liver, 6=brain, and 7=kidney.

FIG. 4

This figure shows Tai-423: NH2-C(DOX-Maleimidyl)-TBP-COOH, wherein its component parts are labeled.

FIGS. 5A an 5B These figures show a cytotoxicity assay (MTT) (PD=peptide-dox). 5,000 cells from the MDA-MB-231 (breast cancer) cell line were seeded in a 96-well plate. Cells were cultured with different concentrations of drugs. At different time points, an MTT agent was added and incubated for 4 hours. A stop reagent was added and incubated overnight. The OD 520 and OD 750 (reference wavelength) were measured.

FIGS. 6A and 6B

These figures show animal experiments employing MDA-MB-231. MDA-MB-231 is a highly aggressive, invasive and poorly differentiated triple-negative breast cancer (TNBC) cell line. This cell line lacks estrogen receptor (ER) and progesterone receptor (PR) expression, as well as HER2 (human epidermal growth factor receptor).

### DETAILED DESCRIPTION OF THE INVENTION

This invention provides conjugates of therapeutic agents and bone-targeting peptides, and methods of using them to treat bone cancer, including primary and metastatic bone cancer.

### Definitions

In this application, certain terms are used which shall have the meanings set forth as follows.

As used herein, “administer”, with respect to an agent, means to deliver the agent to a subject's body via any known method. Specific modes of administration include, without limitation, intravenous, oral, sublingual, transdermal, subcutaneous, intraperitoneal, intramuscular, intrathecal, and intra-tumoral administration, as well as other parenteral administration routes.

In addition, in this invention, the various agents can be formulated using one or more routinely used pharmaceutically acceptable carriers. Such carriers are well known to those skilled in the art. For example, oral delivery systems include tablets and capsules. These can contain excipients such as binders (e.g., hydroxypropylmethylcellulose, polyvinyl pyrilodone, other cellulosic materials and starch), diluents (e.g., lactose, mannitol and other sugars, starch, dicalcium phosphate and cellulosic materials (e.g., carboxymethylcellulose sodium)), disintegrating agents (e.g., starch polymers and cellulosic materials) and lubricating agents (e.g., stearates and talc). Injectable drug delivery systems include, for example, solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs and can contain excipients such as PLGA and polycaprylactone.

As used herein, a “biodegradable bond” between the peptide and therapeutic agent of each subject composition is preferably one having a half-life (i) longer than the time required for the composition to bind to TRAP after administration to a subject, and (ii) shorter than the time by which bone cancer growth is clinically unacceptable. In one embodiment, the biodegradable bond's half-life is one day, two days, three days, four days, five days, six days, seven days, eight days, nine days, 10 days, 11 days, 12 days, 13 days, 14 days, 15 days, 16 days, 17 days, 18 days, three weeks, four weeks, five weeks, six weeks, seven weeks, or eight weeks. In another embodiment, the biodegradable bond's half-life is from one to five days, from five days to 10 days, from 10 days to 20 days, from two weeks to four weeks, and from four weeks to eight weeks. Biodegradable bonds include, without limitation, an ester bond, a thioester bond, an orthoester bond, an amide bond, an anhydride bond, a disulfide bond, or a peptide bond.

As used herein, “bone cancer” includes, without limitation, solid tumors and liquid tumors. Solid tumors include, for example, primary tumors and metastatic tumors (e.g., breast cancer (such as stage IV and triple negative breast cancer), prostate cancer and ovarian cancer). Liquid tumors include, for example, hematologic malignancies such as leukemias (e.g., AML, acute promyelocytic leukemia, acute lymphoblastic leukemia, acute mixed lineage leukemia, chronic myeloid leukemia, chronic lymphocytic leukemia, hairy cell leukemia, large granular lymphocytic leukemia), myelodysplastic syndrome (MDS), myeloproliferative disorders (e.g., polycitermia vera, essential thrombocytosis, primary myelofibrosis and chronic myeloid leukemia), lymphomas, multiple myeloma, and MGUS and similar disorders.

As used herein, a “linker” includes, without limitation, 2,2′-dithiopyridine, oxime, thiazolidine, bosutinib, isothiocyanate, haloacetyl, and maleimide. Preferably, the linker is a maleimide linker. A maleimide linker is relatively stable and hydrolyzes slowly.

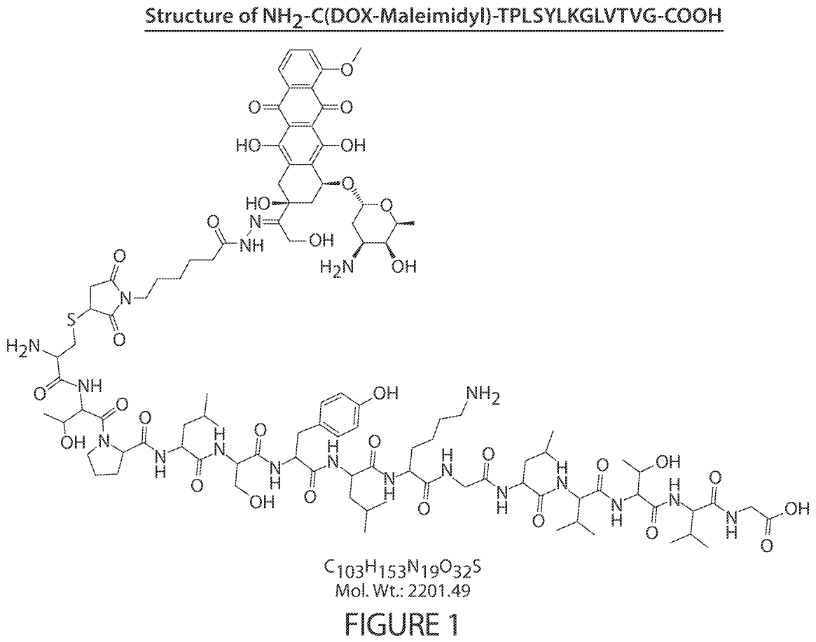

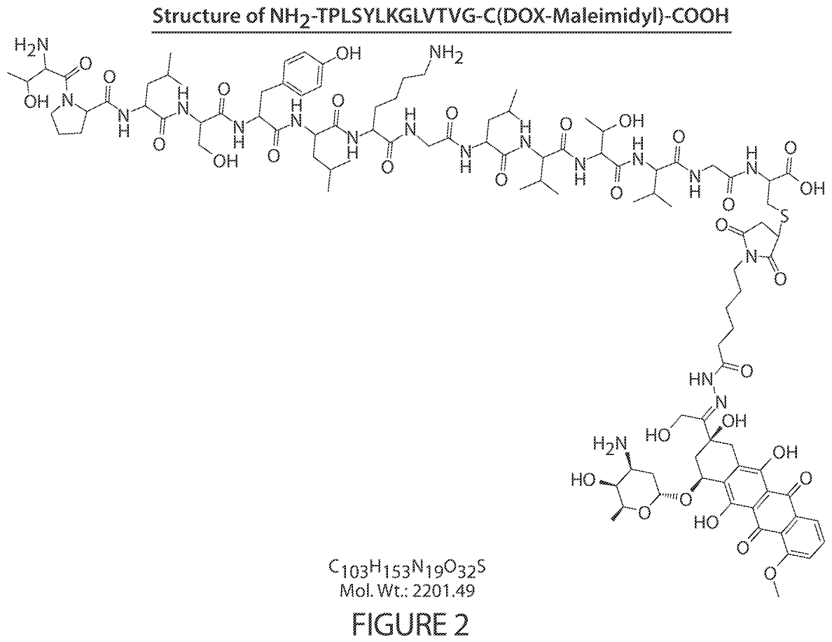

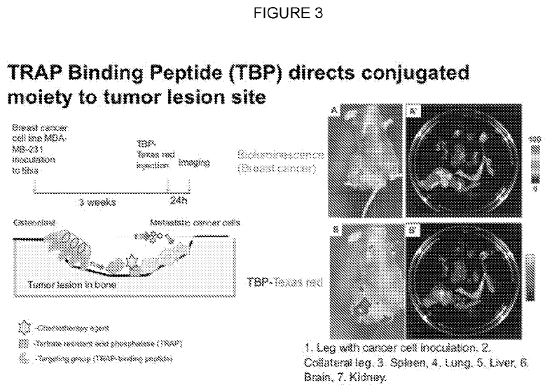

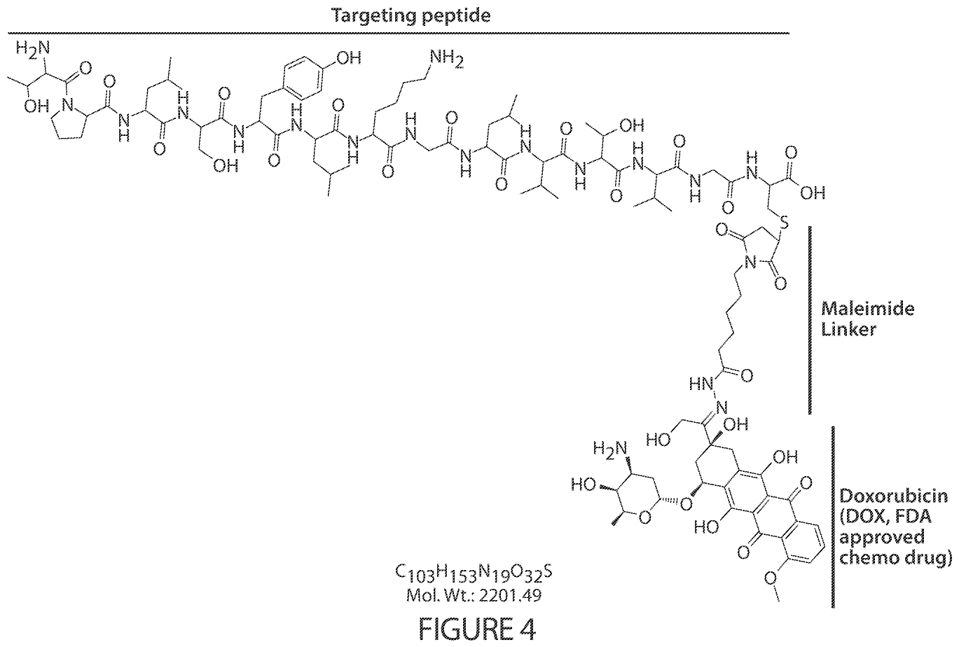

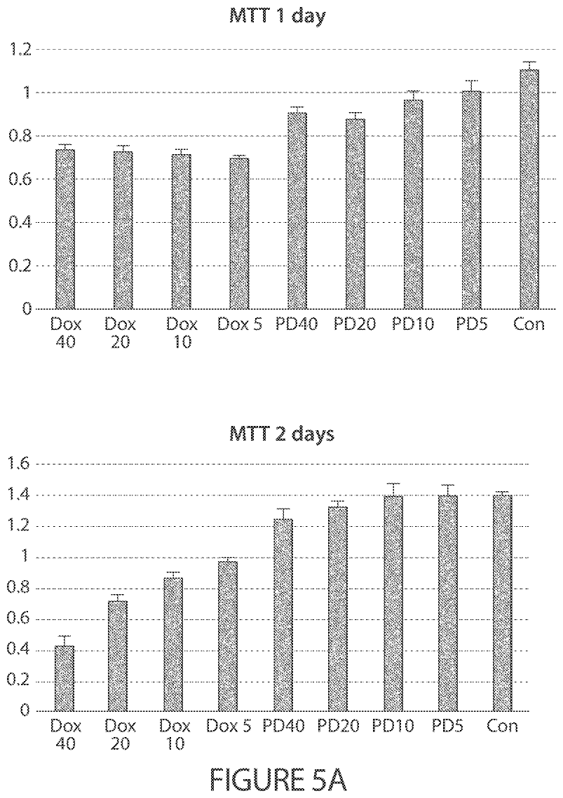

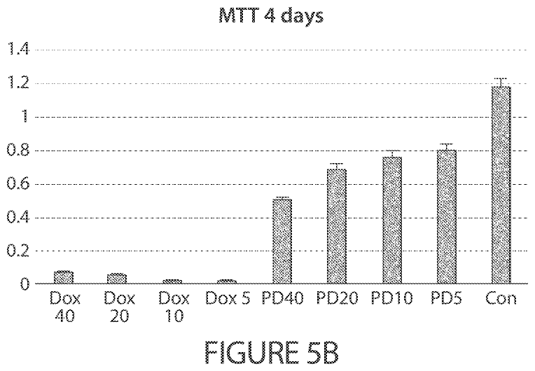

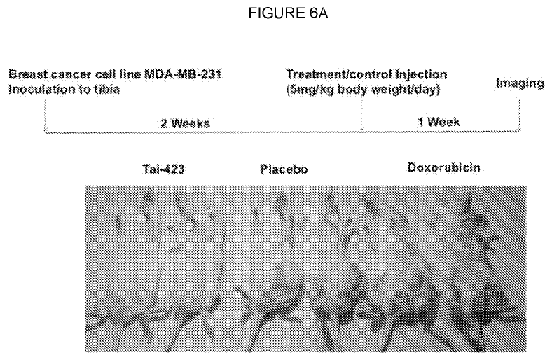

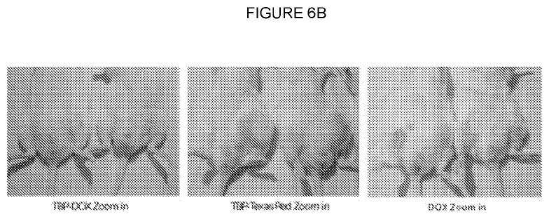

As used herein, the peptide and therapeutic agent of each subject composition are “operably affixed” if (i) the peptide, while part of the composition, can bind to TRAP when contacted therewith, and (ii) the therapeutic agent, while part of the composition, can, once the composition is bound to TRAP, act on bone cancer cells (either while still affixed to the peptide or dissociated therefrom, depending on the agent in question). As an example, the peptide and therapeutic agent (i.e., doxorubicin) in each of the compositions set forth in FIGS. 1 and 2 are operably affixed (in those cases, via a linker), such that the composition's peptide component binds to TRAP in bone, and the composition's doxorubicin component, once it dissociates from the peptide component after TRAP-binding, is free to act on bone cancer cells.

As used herein, the term “subject” includes, without limitation, a mammal such as a human, a non-human primate, a dog, a cat, a horse, a sheep, a goat, a cow, a rabbit, a pig, a rat and a mouse. Preferably, the subject is human. By way of example, the human subject's age can be 50 years or older, 55 years or older, 60 years or older, 65 years or older, 70 years or older, 75 years or older, 80 years or older, or 85 years or older.

As used herein, a “therapeutic agent” is any agent (e.g., a nucleic acid, a protein, a peptide, a small molecule, an aptamer, an antagonist, or a peptidomimetic) intended to ameliorate a disorder and/or its symptoms. Therapeutic agents include, without limitation, anti-cancer drugs, including both small molecule chemotherapeutics (chemotherapeutic agents) and biologics.

Chemothera-peutics include, by way of example, mustard gas derivatives (e.g., mechlorethamine, cyclophosphamide, chlorambucil, melphalan, and ifosfamide); ethylenimines (e.g., thiotepa and hexamethylmelamine); alkylsulfonates (e.g., busulfan); hydrazines and triazines (e.g., altretamine, procarbazine, dacarbazine, and temozolomide); nitrosureas (e.g., carmustine, lomustine, and streptozocin); metal salts (e.g., carboplatin, cisplatin, and oxaliplatin); vinca alkaloids (e.g., vincristine, vinblastine, and vinorelbine); taxanes (e.g., paclitaxel and docetaxel); podophyllotoxins (e.g., etoposide and tenisopide); camptothecan analogs (e.g., irinotecan and topotecan); anthracyclines (e.g., doxorubicin, daunorubicin, epirubicin, mitoxantrone, and idarubicin); chromomycins (e.g., dactinomycin and plicamycin); mitomycin; bleomycin; folic acid antagonists (e.g., methotrexate); pyrimidine antagonists (e.g., 5-fluorouracil, foxuridine, cytarabine, capecitabine, and gemcitabine); purine antagonists (e.g., 6-mercaptopurine and 6-thioguanine); adenosine deaminase inhibitors (e.g., cladribine, fludarabine, nelarabine, and pentostatin); topoisomerase I inhibitors (e.g., ironotecan and topotecan); topoisomerase II inhibitors (e.g., amsacrine, etoposide, etoposide phosphate, and teniposide); ribonucleotide reductase inhibitors (e.g., hydroxyurea); adrenocortical steroid inhibitors (e.g., mitotane); enzymes (e.g., asparaginase and pegaspargase); antimicrotubule agents (e.g., estramustine); and retinoids (e.g., bexarotene, isotretinoin, and tretinoin (ATRA)).

As used herein, the term “therapeutically effective amount”, with respect to the subject composition, refers to the total amount of therapeutic agent present in the dose of composition administered to a subject. In one embodiment, the effective amount is 5 mg, 10 mg, 15 mg, 20 mg, 25 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, or 500 mg. In another embodiment, the effective amount is from 5 mg to 10 mg, from 10 mg to 50 mg, from 50 mg to 100 mg, from 100 mg to 150 mg, from 150 mg to 200 mg, from 200 mg to 250 mg, from 250 mg to 300 mg, from 300 mg to 350 mg, from 350 mg to 400 mg, from 400 mg to 450 mg, or from 450 mg to 500 mg. In a further embodiment, the effective amount is from 1 mg to 250 mg, or from 250 mg to 500 mg. In a still further embodiment, the therapeutically effective amount of the subject composition is an amount that delivers to the subject an amount of the therapeutic agent no more than a defined percentage of the amount of the agent that, alone, is therapeutically effective. So, in this embodiment, the therapeutically effective amount of the subject composition is an amount that delivers, for example, to the subject an amount of the therapeutic agent no more than 5%, 10%, 15%, 20%, 25%, 30%, 35%, 40%, 45%, 50%, 55%, 60%, 65%, 70%, 75%, 80%, 85%, 90%, or 95% of the amount of the agent that, alone, is therapeutically effective. By way of example, assume that the therapeutic agent is doxorubicin, and that the amount of the agent, alone, that is therapeutically effective is 75 mg/m² given intravenously every 21 days. So, in one embodiment, the therapeutically effective amount of the subject composition (given intravenously every 21 days) is an amount that delivers to the subject an amount of the therapeutic agent 25% (i.e., 18.75 mg/m²), 50% (i.e., 37.5 mg/m²), or 75% (i.e., 56.25 mg/m²) of the amount of doxorubicin that, alone, is therapeutically effective.

As used herein, “treating” a subject afflicted with a disorder shall include, without limitation, (i) slowing, stopping or reversing the disorder's progression, (ii) slowing, stopping or reversing the progression of the disorder's symptoms (e.g., pain), (iii) reducing the likelihood of the disorder's recurrence, and/or (iv) reducing the likelihood that the disorder's symptoms will recur. In the preferred embodiment, treating a subject afflicted with a disorder means (i) reversing the disorder's progression, ideally to the point of eliminating the disorder, and/or (ii) reversing the progression of the disorder's symptoms, ideally to the point of eliminating the symptoms.

Embodiments of the Invention

This invention solves an unmet need in the art by providing an unexpectedly superior way to deliver a therapeutic agent, particularly a chemotherapeutic, to bone tissue. The invention does this via affixing the therapeutic agent to a peptide that targets bone tissue by virtue of its affinity to the bone-specific receptor tartrate-resistant acid phosphatase (TRAP) (see, e.g., J. Thundimadathil (“Cancer Treatment Using Peptides: Current Therapies and Future Prospects”, J. Amino Acids, 2012:967347) for a general discussion of peptides as cytotoxic drug carriers).

Specifically, this invention provides a composition comprising (i) a peptide having an amino acid sequence that comprises the amino acid sequence TPLSYLKGLVTV and (ii) a therapeutic agent operably affixed thereto.

In one embodiment of this composition, the peptide has an amino acid sequence comprising the amino acid sequence TPLSYLKGLVTVX, wherein X can be any amino acid residue. Preferably, the peptide has an amino acid sequence that comprises the amino acid sequence TPLSYLKGLVTVG.

In the subject composition, the therapeutic agent can be operably affixed to the peptide either via a linker or via a direct bond with the peptide. Linkers suitable for this purpose include, without limitation, 2,2′-dithiopyridine, oxime, thiazolidine, bosutinib, isothiocyanate, haloacetyl, and maleimide. Preferably, the linker is a maleimide linker.

In the subject composition, the therapeutic agent can be operably affixed to the peptide via the peptide's N-terminal residue or, preferably, the peptide's C-terminal residue. Furthermore, and regardless of whether the therapeutic agent is operably affixed to the peptide via a linker, the agent and peptide can be affixed via any appropriate bond, ideally a biodegradable bond. Biodegradable bonds include, without limitation, an ester bond, a thioester bond, an orthoester bond, an amide bond, an anhydride bond, a disulfide bond, or a peptide bond.

The therapeutic agent envisioned in the subject composition can be any agent whose targeted delivery to bone is desired. In a preferred embodiment, the therapeutic agent is a chemotherapeutic agent such as an anthracycline, and preferably doxorubicin. Other anthracyclines include, without limitation, epirubicin, daunorubicin, and mitoxantrone. Preferably, the doxorubicin-containing composition is the one having either the structure set forth in FIG. 1 or the structure set forth in FIG. 2.

It is envisioned that the subject composition will be administered to a subject in the form of a pharmaceutical composition. Thus, this invention provides a pharmaceutical composition comprising (i) the subject composition and (ii) a pharmaceutically acceptable carrier.

This invention further provides a method for treating a subject (preferably human) afflicted with bone cancer comprising administering to the subject a therapeutically effective amount of the subject composition.

Although the bone cancer treated by the subject method can be either solid or liquid, the bone cancer is preferably a tumor. This tumor, in turn, can be a primary bone tumor or, preferably, a metastatic bone tumor (i.e., one originating from a tumor in a different tissue, such as a breast tumor).

One advantage of the subject method is that it permits treating bone cancer by administering less therapeutic agent to a subject than would be necessary if the agent were not affixed to a bone-targeting peptide. Ideally, this reduces side effects. Accordingly, in one embodiment, the therapeutically effective amount of the subject composition is an amount that delivers to the subject an amount of the therapeutic agent equal to or less than the amount of the agent that, alone, is therapeutically effective. In another embodiment, the therapeutically effective amount of the subject composition is an amount that delivers to the subject an amount of the therapeutic agent no more than 50% of the amount of the agent that, alone, is therapeutically effective. In a further embodiment, the therapeutically effective amount of the subject composition is an amount that delivers to the subject an amount of the therapeutic agent no more than 25% of the amount of the agent that, alone, is therapeutically effective.

The subject method can be practiced either with or without concurrently administering to the subject a second therapeutic agent for treating bone cancer. In one embodiment, the subject method (e.g., wherein the first therapeutic agent is doxorubicin) further comprises administering to the subject a second therapeutic agent for treating bone cancer. Second therapeutic agents include, without limitation, other chemotherapy drugs (e.g., docetaxel, cyclophosphamide, and epirubicin); HER2 receptor inhibitors (e.g., trastuzumab); osteoporosis drugs (e.g., bisphosphonates, and romosozumab); and checkpoint inhibitors (e.g., PD-1, PDL-1, and CTLA-4 blockers such as nivolumab, pembrolizumab). In a further embodiment, the subject method comprises administering to the subject an admixture of the compositions of FIGS. 1 and 2.

Finally, this invention provides an article of manufacture (kit) comprising, in separate compartments, (a) one of, and ideally both of, (i) a diluent and (ii) a label instructing the user to administer a composition to a subject afflicted with bone cancer, and (b) the composition, wherein the composition comprises (i) a peptide having an amino acid sequence that comprises the amino acid sequence TPLSYLKGLVTV (preferably TPLSYLKGLVTV or TPLSYLKGLVTVG) and (ii) a therapeutic agent (preferably a chemotherapeutic agent such as doxorubicin) operably affixed thereto (preferably via a linker such as a maleimide linker). Where applicable, the embodiments described above for the instant compositions and methods are also envisioned for this article of manufacture.

This invention will be better understood by reference to the examples set forth in the figures, but those skilled in the art will readily appreciate that the specific examples detailed are only illustrative of the invention as described more fully in the claims.

1. A composition comprising (i) a peptide having an amino acid sequence that comprises the amino acid sequence TPLSYLKGLVTV (SEQ ID NO:1) and (ii) a therapeutic agent operably affixed thereto.

2. The composition of claim 1, wherein the peptide has an amino acid sequence that comprises the amino acid sequence TPLSYLKGLVTVG (SEQ ID NO:3).

3. The composition of claim 1, wherein the therapeutic agent is operably affixed to the peptide via a linker.

4. The composition of claim 3, wherein the linker is a maleimide linker.

5. The composition of claim 1, wherein the therapeutic agent is operably affixed to the peptide via the peptide's C-terminal residue.

6. The composition of claim 1, wherein the therapeutic agent is operably affixed to the peptide via a biodegradable bond selected from the group consisting of an ester bond, a thioester bond, an orthoester bond, an amide bond, an anhydride bond, a disulfide bond, and a peptide bond.

7. The composition of claim 1, wherein the therapeutic agent is a chemotherapeutic agent.

8. The composition of claim 7, wherein the chemotherapeutic agent is an anthracycline.

9. The composition of claim 8, wherein the chemotherapeutic agent is doxorubicin.

10. The composition of claim 1, wherein the composition has the structure set forth in FIG. 1.

11. The composition of claim 1 any of claim 1, wherein the composition has the structure set forth in FIG. 2.

12. A composition comprising (i) the composition of claim 1 and (ii) a pharmaceutically acceptable carrier.

13. A method for treating a subject afflicted with bone cancer comprising administering to the subject a therapeutically effective amount of the composition of claim 1.

14. The method of claim 13, wherein the subject is a human.

15. The method of claim 13, wherein the bone cancer is a primary bone tumor.

16. The method of claim 13, wherein the bone tumor is a metastatic bone tumor.

17. The method of claim 16, wherein the metastatic bone tumor originated from a breast tumor.

18. The method of claim 13, wherein the therapeutically effective amount of the composition is an amount that delivers to the subject an amount of the therapeutic agent equal to or less than the amount of the agent that, alone, is therapeutically effective.

19. The method of claim 18, wherein the therapeutically effective amount of the composition is an amount that delivers to the subject an amount of the therapeutic agent no more than 50% of the amount of the agent that, alone, is therapeutically effective.

20. The method of claim 19, wherein the therapeutically effective amount of the composition is an amount that delivers to the subject an amount of the therapeutic agent no more than 25% of the amount of the agent that, alone, is therapeutically effective.

21. The method of claim 13, further comprising concurrently administering to the subject a second therapeutic agent for treating bone cancer.


SEQUENCE LISTING (5 sequences)

SEQ ID NO: 1 - protein, 12 aa, Artificial Sequence
  Description of Artificial Sequence Synthetic peptide
TPLSYLKGLVTV

SEQ ID NO: 2 - protein, 13 aa, Artificial Sequence
  Description of Artificial Sequence Synthetic peptide
  MOD_RES (13)..(13): Any amino acid
TPLSYLKGLVTVX

SEQ ID NO: 3 - protein, 13 aa, Artificial Sequence
  Description of Artificial Sequence Synthetic peptide
TPLSYLKGLVTVG

SEQ ID NO: 4 - protein, 14 aa, Artificial Sequence
  Description of Artificial Sequence Synthetic peptide
  MOD_RES (1)..(1): Cys(DOX-maleimidyl)
CTPLSYLKGLVTVG

SEQ ID NO: 5 - protein, 14 aa, Artificial Sequence
  Description of Artificial Sequence Synthetic peptide
  MOD_RES (14)..(14): Cys(DOX-maleimidyl)
TPLSYLKGLVTVGC